(19) United States
(12) Patent Application Publication
(10) Pub. No.: US 2023/0091124 A1
(43) Pub. Date: Mar. 23, 2023

(54) NOISE REDUCTION IN NON-INVASIVE RADIO FREQUENCY ANALYTE SENSORS

(71) Applicant: Know Labs, Inc., Seattle, WA (US)

(72) Inventor: Phillip BOSUA, Seattle, WA (US)

(21) Appl. No.: 17/478,084

(22) Filed: Sep. 17, 2021

(51) Int. Cl. A61B 5/145; H04B 1/10; A61B 5/1477

(52) U.S. Cl. CPC A61B 5/14532; H04B 1/1018; H04B 1/1009; A61B 5/1477

(57) ABSTRACT

A non-invasive analyte sensor that includes one or more noise reduction components provided on the receive and/or transmit components to reduce extraneous radio frequency noise. A noise reduction component can be provided on the exterior of an electrical conductor that connects the receive antenna with the receive circuitry to suppress extraneous radio frequency noise that is generated on the exterior of the electrical conductor. The noise reduction component can be any type of noise reduction device that achieves such radio frequency noise suppression. In one embodiment, the noise reduction component can be a choke.

### FIELD

This disclosure relates generally to apparatus, systems and methods of detecting an analyte via spectroscopic techniques using an analyte sensor that includes a detector array (also referred to as an antenna array), wherein the detector array operates in the radio or microwave frequency range of the electromagnetic spectrum, and reducing noise in the analyte sensor.

### BACKGROUND

There is interest in being able to detect and/or measure an analyte within a target. One example is measuring glucose in biological material. In the example of measuring glucose in a patient, current analyte measurement methods are invasive in that they perform the measurement on a bodily fluid such as blood for fingerstick or laboratory-based tests, or on fluid that is drawn from the patient often using an invasive transcutaneous device. There are non-invasive methods that claim to be able to perform glucose measurements in biological material. However, many of the non-invasive methods generally suffer from: lack of specificity to the analyte of interest, such as glucose; interference from temperature fluctuations; interference from skin compounds (i.e. sweat) and pigments; and complexity of placement, i.e. the sensing device resides on multiple locations on the patient's body.

### SUMMARY

This disclosure relates generally to apparatus, systems and methods of detecting an analyte via spectroscopic techniques using non-optical frequencies such as in the radio or microwave frequency range of the electromagnetic spectrum. An analyte sensor described herein includes a detector array having a plurality of detector elements (also referred to as antenna elements or antennas) at least one of which can transmit an electromagnetic signal in the radio or microwave frequency range and at least one of which can receive an electromagnetic signal in the radio or microwave frequency range resulting from transmission of the electromagnetic signal.

In the non-invasive analyte sensor described herein, noise reduction components are provided on the receive and/or transmit components to reduce extraneous radio frequency noise. In one embodiment, a noise reduction component is provided on the exterior of an electrical conductor that connects the receive antenna with the receive circuitry to suppress extraneous radio frequency noise that is generated on the exterior of the electrical conductor. The noise reduction component can be any type of noise reduction device that achieves such radio frequency noise suppression. In one embodiment, the noise reduction component can be a choke.

The noise reduction element(s) described herein is distinct from a band-pass filter or other filter that is present in a signal transmission path. The noise reduction element(s) described herein is external to the signal transmission path and separate from any other electronic components that may be on and part of the signal transmission path. In one embodiment, the noise reduction element(s) can be external to the signal transmission path, such as on the exterior of an electrical conductor or on the exterior of a radio frequency adapter (also referred to as a bullet adapter) that connects the electrical conductor to an antenna and/or to the transmit or receive circuit.

In one embodiment described herein, a non-invasive analyte sensor system can include a first antenna that is configured to emit radio frequency electromagnetic waves, where the first antenna is positioned and arranged to transmit a radio frequency transmit signal into a target containing at least one analyte, and a second antenna that is configured to detect radio frequency electromagnetic waves, where the second antenna is positioned and arranged to detect a radio frequency response resulting from transmission of the radio frequency transmit signal by the first antenna into the target containing the at least one analyte. A transmit circuit is electrically connectable to the first antenna, where the transmit circuit is configured to generate the radio frequency transmit signal to be transmitted by the first antenna, and a transmit electrical conductor electrically connects the transmit circuit with the first antenna. In addition, a receive circuit is electrically connectable to the second antenna, where the receive circuit is configured to receive the radio frequency response detected by the second antenna, and a receive electrical conductor electrically connects the receive circuit with the second antenna. At least one noise reducer is on the receive electrical conductor between the receive circuit and the second antenna and/or on the transmit electrical conductor between the transmit circuit and the first antenna.

In an embodiment, the noise reducer can be on the outside of the receive electrical conductor and that reduces at least 30-50% of stray radio frequency energy in the receive electrical conductor.

In another embodiment described herein, a non-invasive analyte sensor system can include an antenna array having at least three antennas each of which is configured to emit and receive radio frequency electromagnetic waves. A transmit circuit is selectively electrically connectable to any one or more of the at least three antennas, where the transmit circuit is configured to generate at least one transmit signal in a radio frequency range of the electromagnetic spectrum to be transmitted into the target by the one or more of the at least three antennas the transmit circuit is electrically connected to. A receive circuit is selectively electrically connectable to any one or more of the at least three antennas, where the receive circuit is configured to receive a response detected by the one or more of the at least three antennas the receive circuit is electrically connected to resulting from transmission of the at least one transmit signal into the target containing the at least one analyte of interest. In addition, electrical conductors electrically connect the receive circuit with the at least three antennas, and noise reducers are on the electrical conductors between the receive circuit and the at least three antennas.

### DRAWINGS

FIG. 1 is a schematic depiction of an analyte sensor system with an analyte sensor relative to a target according to an embodiment.

FIG. 2 is a schematic depiction of an electrical conductor connecting the receive antenna/element and the receive circuit.

FIG. 3 is a schematic depiction of an electrical conductor connecting the transmit antenna/element and the transmit circuit.

FIG. 4 is a schematic depiction of an embodiment of a non-invasive analyte sensor system with an antenna array having three antennas.

FIG. 5 is a schematic depiction of another embodiment of a non-invasive analyte sensor system with an antenna array having six antennas.

Like reference numbers represent like parts throughout.

### DETAILED DESCRIPTION

The following is a detailed description of apparatus, systems and methods of detecting an analyte via spectroscopic techniques using non-optical frequencies such as in the radio or microwave frequency bands of the electromagnetic spectrum. An analyte sensor described herein includes a detector array having a plurality of detector elements (also referred to as antenna elements or antennas) at least one of which can transmit an electromagnetic signal in the radio or microwave frequency range and at least one of which can receive an electromagnetic signal in the radio or microwave frequency range resulting from transmission of the electromagnetic signal. For sake of convenience, the detector array will hereinafter be referred to as an antenna array and the detector elements will hereinafter be referred to as antennas.

In one embodiment, the sensor systems described herein can be used to detect the presence of at least one analyte in a target. In another embodiment, the sensor systems described herein can detect an amount or a concentration of the at least one analyte in the target. The target can be any target containing at least one analyte of interest that one may wish to detect. The target can be human or non-human, animal or non-animal, biological or non-biological. For example, the target can include, but is not limited to, human tissue, animal tissue, plant tissue, an inanimate object, soil, a fluid, genetic material, or a microbe. Non-limiting examples of targets include, but are not limited to, a fluid, for example blood, interstitial fluid, cerebral spinal fluid, lymph fluid or urine, human tissue, animal tissue, plant tissue, an inanimate object, soil, genetic material, or a microbe.

The detection by the sensors described herein can be non-invasive meaning that the sensor remains outside the target, such as the human body, and the detection of the analyte occurs without requiring removal of fluid or other removal from the target, such as the human body. In the case of sensing in the human body, this non-invasive sensing may also be referred to as in vivo sensing. In other embodiments, the sensors described herein may be an in vitro sensor where the material containing the analyte has been removed, for example from a human body.

The transmit antenna and the receive antenna can be located near the target and operated as further described herein to assist in detecting at least one analyte in the target. The transmit antenna transmits a signal, which has at least two frequencies in the radio or microwave frequency range, toward and into the target. The signal with the at least two frequencies can be formed by separate signal portions, each having a discrete frequency, that are transmitted separately at separate times at each frequency. In another embodiment, the signal with the at least two frequencies may be part of a complex signal that includes a plurality of frequencies including the at least two frequencies. The complex signal can be generated by blending or multiplexing multiple signals together followed by transmitting the complex signal whereby the plurality of frequencies are transmitted at the same time. One possible technique for generating the complex signal includes, but is not limited to, using an inverse Fourier transformation technique. The receive antenna detects a response resulting from transmission of the signal by the transmit antenna into the target containing the at least one analyte of interest.

The transmit antenna and the receive antenna may be decoupled (which may also be referred to as detuned or the like) from one another. Decoupling refers to intentionally fabricating the configuration and/or arrangement of the transmit antenna and the receive antenna to minimize direct communication between the transmit antenna and the receive antenna, preferably absent shielding. Shielding between the transmit antenna and the receive antenna can be utilized. However, the transmit antenna and the receive antenna are decoupled even without the presence of shielding.

The signal(s) detected by the receive antenna can be analyzed to detect the analyte based on the intensity of the received signal(s) and reductions in intensity at one or more frequencies where the analyte absorbs the transmitted signal. An example of detecting an analyte using a non-invasive spectroscopy sensor operating in the radio or microwave frequency range of the electromagnetic spectrum is described in U.S. Pat. No. 10,548,503, the entire contents of which are incorporated herein by reference. The signal(s) detected by the receive antenna can be complex signals including a plurality of signal components, each signal component being at a different frequency. In an embodiment, the detected complex signals can be decomposed into the signal components at each of the different frequencies, for example through a Fourier transformation. In an embodiment, the complex signal detected by the receive antenna can be analyzed as a whole (i.e. without demultiplexing the complex signal) to detect the analyte as long as the detected signal provides enough information to make the analyte detection. In addition, the signal(s) detected by the receive antenna can be separate signal portions, each having a discrete frequency.

The analyte(s) can be any analyte that one may wish to detect. The analyte can be human or non-human, animal or non-animal, biological or non-biological. For example, the analyte(s) can include, but is not limited to, one or more of blood glucose, blood alcohol, white blood cells, or luteinizing hormone. The analyte(s) can include, but is not limited to, a chemical, a combination of chemicals, a virus, a bacteria, or the like. The analyte can be a chemical included in another medium, with non-limiting examples of such media including a fluid containing the at least one analyte, for example blood, interstitial fluid, cerebral spinal fluid, lymph fluid or urine, human tissue, animal tissue, plant tissue, an inanimate object, soil, genetic material, or a microbe. The analyte(s) may also be a non-human, non-biological particle such as a mineral or a contaminant.

The analyte(s) can include, for example, naturally occurring substances, artificial substances, metabolites, and/or reaction products. As non-limiting examples, the at least one analyte can include, but is not limited to, insulin, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); androstenedione; antipyrine; arabinitol enantiomers; arginase; benzoylecgonine (cocaine); biotinidase; biopterin; c-reactive protein; carnitine; pro-BNP; BNP; troponin; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione peroxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, polio virus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin.

The analyte(s) can also include one or more chemicals introduced into the target. The analyte(s) can include a marker such as a contrast agent, a radioisotope, or other chemical agent. The analyte(s) can include a fluorocarbon-based synthetic blood. The analyte(s) can include a drug or pharmaceutical composition, with non-limiting examples including ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The analyte(s) can include other drugs or pharmaceutical compositions. The analyte(s) can include neurochemicals or other chemicals generated within the body, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA).

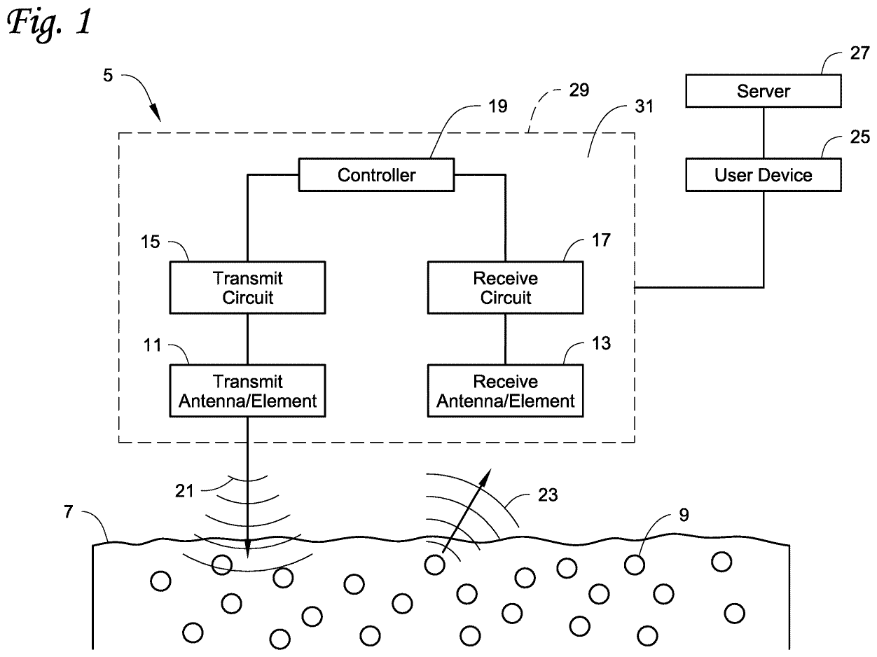

Referring now to FIG. 1, an embodiment of a non-invasive analyte sensor system with a non-invasive analyte sensor 5 is illustrated. The sensor 5 is depicted relative to a target 7 that contains an analyte of interest 9, for example an analyte in interstitial fluid in a human body. In this example, the sensor 5 is depicted as including an antenna array that includes a transmit antenna/element 11 (hereinafter “transmit antenna 11”) and a receive antenna/element 13 (hereinafter “receive antenna 13”). The sensor 5 further includes a transmit circuit 15, a receive circuit 17, and a controller 19. As discussed further below, the sensor 5 can also include a power supply, such as a battery (not shown in FIG. 1). In some embodiments, power can be provided from mains power, for example by plugging the sensor 5 into a wall socket via a cord connected to the sensor 5.

The transmit antenna 11 is positioned, arranged and configured to transmit a signal 21 that is the radio frequency (RF) or microwave range of the electromagnetic spectrum into the target 7. The transmit antenna 11 can be an electrode or any other suitable transmitter of electromagnetic signals in the radio frequency (RF) or microwave range. The transmit antenna 11 can have any arrangement and orientation relative to the target 7 that is sufficient to allow the analyte sensing to take place. In one non-limiting embodiment, the transmit antenna 11 can be arranged to face in a direction that is substantially toward the target 7.

The signal 21 transmitted by the transmit antenna 11 is generated by the transmit circuit 15 which is electrically connectable to the transmit antenna 11. The transmit circuit 15 can have any configuration that is suitable to generate a transmit signal to be transmitted by the transmit antenna 11. Transmit circuits for generating transmit signals in the RF or microwave frequency range are well known in the art. In one embodiment, the transmit circuit 15 can include, for example, a connection to a power source, a frequency generator, and optionally filters, amplifiers or any other suitable elements for a circuit generating an RF or microwave frequency electromagnetic signal. In an embodiment, the signal generated by the transmit circuit 15 can have at least two discrete frequencies (i.e. a plurality of discrete frequencies), each of which is in the range from about 10 kHz to about 100 GHz. In another embodiment, each of the at least two discrete frequencies can be in a range from about 300 MHz to about 6000 MHz. In an embodiment, the transmit circuit 15 can be configured to sweep through a range of frequencies that are within the range of about 10 kHz to about 100 GHz, or in another embodiment a range of about 300 MHz to about 6000 MHz. In an embodiment, the transmit circuit 15 can be configured to produce a complex transmit signal, the complex signal including a plurality of signal components, each of the signal components having a different frequency. The complex signal can be generated by blending or multiplexing multiple signals together followed by transmitting the complex signal whereby the plurality of frequencies are transmitted at the same time.

The receive antenna 13 is positioned, arranged, and configured to detect one or more electromagnetic response signals 23 that result from the transmission of the transmit signal 21 by the transmit antenna 11 into the target 7 and impinging on the analyte 9. The receive antenna 13 can be an electrode or any other suitable receiver of electromagnetic signals in the radio frequency (RF) or microwave range. In an embodiment, the receive antenna 13 is configured to detect electromagnetic signals having at least two frequencies, each of which is in the range from about 10 kHz to about 100 GHz, or in another embodiment a range from about 300 MHz to about 6000 MHz. The receive antenna 13 can have any arrangement and orientation relative to the target 7 that is sufficient to allow detection of the response signal(s) 23 to allow the analyte sensing to take place. In one non-limiting embodiment, the receive antenna 13 can be arranged to face in a direction that is substantially toward the target 7.

The receive circuit 17 is electrically connectable to the receive antenna 13 and conveys the received response from the receive antenna 13 to the controller 19. The receive circuit 17 can have any configuration that is suitable for interfacing with the receive antenna 13 to convert the electromagnetic energy detected by the receive antenna 13 into one or more signals reflective of the response signal(s) 23. The construction of receive circuits are well known in the art. The receive circuit 17 can be configured to condition the signal(s) prior to providing the signal(s) to the controller 19, for example through amplifying the signal(s), filtering the signal(s), or the like. Accordingly, the receive circuit 17 may include filters, amplifiers, or any other suitable components for conditioning the signal(s) provided to the controller 19. In an embodiment, at least one of the receive circuit 17 or the controller 19 can be configured to decompose or demultiplex a complex signal, detected by the receive antenna 13, including a plurality of signal components each at different frequencies into each of the constituent signal components. In an embodiment, decomposing the complex signal can include applying a Fourier transform to the detected complex signal. However, decomposing or demultiplexing a received complex signal is optional. Instead, in an embodiment, the complex signal detected by the receive antenna can be analyzed as a whole (i.e. without demultiplexing the complex signal) to detect the analyte as long as the detected signal provides enough information to make the analyte detection.

The controller 19 controls the operation of the sensor 5. The controller 19, for example, can direct the transmit circuit 15 to generate a transmit signal to be transmitted by the transmit antenna 11. The controller 19 further receives signals from the receive circuit 17. The controller 19 can optionally process the signals from the receive circuit 17 to detect the analyte(s) 9 in the target 7. In one embodiment, the controller 19 may optionally be in communication with at least one external device 25 such as a user device and/or a remote server 27, for example through one or more wireless connections such as Bluetooth, wireless data connections such a 4G, 5G, LTE or the like, or Wi-Fi. If provided, the external device 25 and/or remote server 27 may process (or further process) the signals that the controller 19 receives from the receive circuit 17, for example to detect the analyte(s) 9. If provided, the external device 25 may be used to provide communication between the sensor 5 and the remote server 27, for example using a wired data connection or via a wireless data connection or Wi-Fi of the external device 25 to provide the connection to the remote server 27.

With continued reference to FIG. 1, the sensor 5 may include a sensor housing 29 (shown in dashed lines) that defines an interior space 31. Components of the sensor 5 may be attached to and/or disposed within the housing 29. For example, the transmit antenna 11 and the receive antenna 13 are attached to the housing 29. In some embodiments, the antennas 11, 13 may be entirely or partially within the interior space 31 of the housing 29. In some embodiments, the antennas 11, 13 may be attached to the housing 29 but at least partially or fully located outside the interior space 31. In some embodiments, the transmit circuit 15, the receive circuit 17 and the controller 19 are attached to the housing 29 and disposed entirely within the sensor housing 29.

The receive antenna 13 may be decoupled or detuned with respect to the transmit antenna 11 such that electromagnetic coupling between the transmit antenna 11 and the receive antenna 13 is reduced. The decoupling of the transmit antenna 11 and the receive antenna 13 increases the portion of the signal(s) detected by the receive antenna 13 that is the response signal(s) 23 from the target 7, and minimizes direct receipt of the transmitted signal 21 by the receive antenna 13. The decoupling of the transmit antenna 11 and the receive antenna 13 results in transmission from the transmit antenna 11 to the receive antenna 13 having a reduced forward gain (S₂₁) and an increased reflection at output (S₂₂) compared to antenna systems having coupled transmit and receive antennas.

In an embodiment, coupling between the transmit antenna 11 and the receive antenna 13 is 95% or less. In another embodiment, coupling between the transmit antenna 11 and the receive antenna 13 is 90% or less. In another embodiment, coupling between the transmit antenna 11 and the receive antenna 13 is 85% or less. In another embodiment, coupling between the transmit antenna 11 and the receive antenna 13 is 75% or less.

Any technique for reducing coupling between the transmit antenna 11 and the receive antenna 13 can be used. For example, the decoupling between the transmit antenna 11 and the receive antenna 13 can be achieved by one or more intentionally fabricated configurations and/or arrangements between the transmit antenna 11 and the receive antenna 13 that is sufficient to decouple the transmit antenna 11 and the receive antenna 13 from one another.

For example, in one embodiment described further below, the decoupling of the transmit antenna 11 and the receive antenna 13 can be achieved by intentionally configuring the transmit antenna 11 and the receive antenna 13 to have different geometries from one another. Intentionally different geometries refers to different geometric configurations of the transmit and receive antennas 11, 13 that are intentional. Intentional differences in geometry are distinct from differences in geometry of transmit and receive antennas that may occur by accident or unintentionally, for example due to manufacturing errors or tolerances.

Another technique to achieve decoupling of the transmit antenna 11 and the receive antenna 13 is to provide appropriate spacing between each antenna 11, 13 that is sufficient to decouple the antennas 11, 13 and force a proportion of the electromagnetic lines of force of the transmitted signal 21 into the target 7 thereby minimizing or eliminating as much as possible direct receipt of electromagnetic energy by the receive antenna 13 directly from the transmit antenna 11 without traveling into the target 7. The appropriate spacing between each antenna 11, 13 can be determined based upon factors that include, but are not limited to, the output power of the signal from the transmit antenna 11, the size of the antennas 11, 13, the frequency or frequencies of the transmitted signal, and the presence of any shielding between the antennas. This technique helps to ensure that the response detected by the receive antenna 13 is measuring the analyte 9 and is not just the transmitted signal 21 flowing directly from the transmit antenna 11 to the receive antenna 13. In some embodiments, the appropriate spacing between the antennas 11, 13 can be used together with the intentional difference in geometries of the antennas 11, 13 to achieve decoupling.

In one embodiment, the transmit signal that is transmitted by the transmit antenna 11 can have at least two different frequencies, for example upwards of 7 to 12 different and discrete frequencies. In another embodiment, the transmit signal can be a series of discrete, separate signals with each separate signal having a single frequency or multiple different frequencies.

In one embodiment, the transmit signal (or each of the transmit signals) can be transmitted over a transmit time that is less than, equal to, or greater than about 300 ms. In another embodiment, the transmit time can be than, equal to, or greater than about 200 ms. In still another embodiment, the transmit time can be less than, equal to, or greater than about 30 ms. The transmit time could also have a magnitude that is measured in seconds, for example 1 second, 5 seconds, 10 seconds, or more. In an embodiment, the same transmit signal can be transmitted multiple times, and then the transmit time can be averaged. In another embodiment, the transmit signal (or each of the transmit signals) can be transmitted with a duty cycle that is less than or equal to about 50%.

The interaction between the transmitted signal and the analyte may, in some cases, increase the intensity of the signal(s) that is detected by the receive antenna, and may, in other cases, decrease the intensity of the signal(s) that is detected by the receive antenna. For example, in one non-limiting embodiment, when analyzing the detected response, compounds in the target, including the analyte of interest that is being detected, can absorb some of the transmit signal, with the absorption varying based on the frequency of the transmit signal. The response signal detected by the receive antenna may include drops in intensity at frequencies where compounds in the target, such as the analyte, absorb the transmit signal. The frequencies of absorption are particular to different analytes. The response signal(s) detected by the receive antenna can be analyzed at frequencies that are associated with the analyte of interest to detect the analyte based on drops in the signal intensity corresponding to absorption by the analyte based on whether such drops in signal intensity are observed at frequencies that correspond to the absorption by the analyte of interest. A similar technique can be employed with respect to increases in the intensity of the signal(s) caused by the analyte.

Detection of the presence of the analyte can be achieved, for example, by identifying a change in the signal intensity detected by the receive antenna at a known frequency associated with the analyte. The change may be a decrease in the signal intensity or an increase in the signal intensity depending upon how the transmit signal interacts with the analyte. The known frequency associated with the analyte can be established, for example, through testing of solutions known to contain the analyte. Determination of the amount of the analyte can be achieved, for example, by identifying a magnitude of the change in the signal at the known frequency, for example using a function where the input variable is the magnitude of the change in signal and the output variable is an amount of the analyte. The determination of the amount of the analyte can further be used to determine a concentration, for example based on a known mass or volume of the target. In an embodiment, presence of the analyte and determination of the amount of analyte may both be determined, for example by first identifying the change in the detected signal to detect the presence of the analyte, and then processing the detected signal(s) to identify the magnitude of the change to determine the amount.

Further information on the sensor 5 and its components and variations thereof can be found in U.S. Pat. Nos. 11,063,373, 11,031,970, 11,058,317, 11,058,331 and 11,033,208 the entire contents of which are incorporated herein by reference in their entirety.

The sensor 5 includes one or more noise reduction components on the receive and/or transmit components to reduce extraneous radio frequency noise. The noise reduction component(s) described herein is external to the signal transmission path between the receive antenna 13 and the receive circuit 17 and the transmit antenna 11 and the transmit circuit 15, and separate from any other electronic components that may be on and part of the signal transmission path. The noise reduction component(s) described herein is distinct from a band-pass filter or other filter that is present in a signal transmission path. The noise reduction element(s) described herein is external to the signal transmission path and separate from any other electronic components that may be on and part of the signal transmission path.

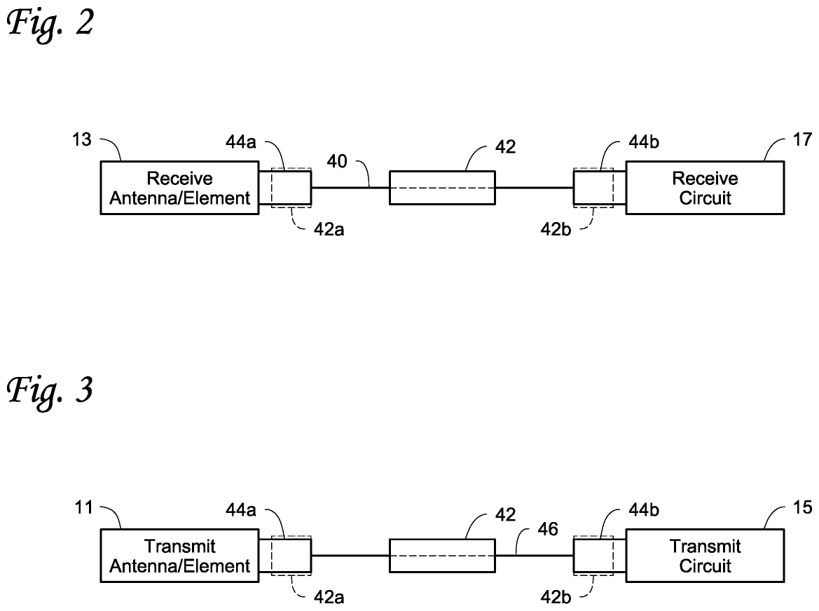

Referring to FIG. 2, one embodiment is illustrated. In this embodiment, the receive antenna 13 is schematically illustrated as being electrically connected to the receive circuit 17 via an electrical conductor 40 (which may be also be referred to as a cable or wire). The conductor 40 provides a signal transmission path between the receive antenna 13 and the receive circuit 17 to direct a signal detected by the receive antenna 13 to the receive circuit 17. The conductor 40 can form a direct signal path between the receive antenna 13 and the receive circuit 17, or intermediate electrical components (not illustrated) can be provided on the signal path between the receive antenna 13 and the receive circuit 17.

One or more noise reduction components 42 are provided on the exterior of the conductor 40. The noise reduction component 42 suppresses extraneous radio frequency noise that is generated on the exterior of the electrical conductor 40. The noise reduction component 42 can be any type of noise reduction device that achieves such radio frequency noise suppression. In one embodiment, the noise reduction component 42 can be a choke. An example of a suitable choke that can be used is available from Laird Technologies, Inc. of Chesterfield, Mo.

The noise reduction component(s) 42 can be provided anywhere on the outside of the conductor 40 sufficient to suppress extraneous radio frequency noise. For example, in one embodiment, the noise reduction component 42 can be provided on the exterior of the conductor 40 between the receive antenna 13 and the receive circuit 17.

FIG. 2 also illustrates that the conductor 40 may optionally be connected to the receive antenna 13 and to the receive circuit 17 via radio frequency (RF) adapters 44a, 44b, respectively. The adapters 44a, 44b (shown in solid lines in FIG. 2) may also be referred to as bullet adapters. As depicted in FIG. 2, in one embodiment, noise reduction components 42a, 42b (shown in dashed/broken lines), such as chokes, may be provided on the outside of the adapters 44a, 44b to suppress extraneous RF noise. In another embodiment, all of the noise reduction components 42, 42a, 42b may be utilized. In an embodiment, the noise reduction component(s) 42, 42a, 42b reduce RF noise by about 30% to about 50%.

Another embodiment is illustrated in FIG. 3. In FIG. 3, elements that are similar to elements in FIG. 2 are referenced using the same reference numerals. In this embodiment, the transmit antenna 11 is schematically illustrated as being electrically connected to the transmit circuit 15 via an electrical conductor 46 (which may also be referred to as a cable or wire). The conductor 46 provides a signal transmission path between the transmit antenna 11 and the transmit circuit 15 to direct a signal to be transmitted by the transmit antenna 11 from the transmit circuit 15. The conductor 46 can form a direct signal path between the transmit antenna 11 and the transmit circuit 15, or intermediate electrical components (not illustrated) can be provided on the signal path between the transmit antenna 11 and the transmit circuit 15. One or more of the noise reduction components 42 may be provided on the exterior of the conductor 46 and/or on the outside of the RF adapters 44a, 44b to suppress extraneous RF noise.

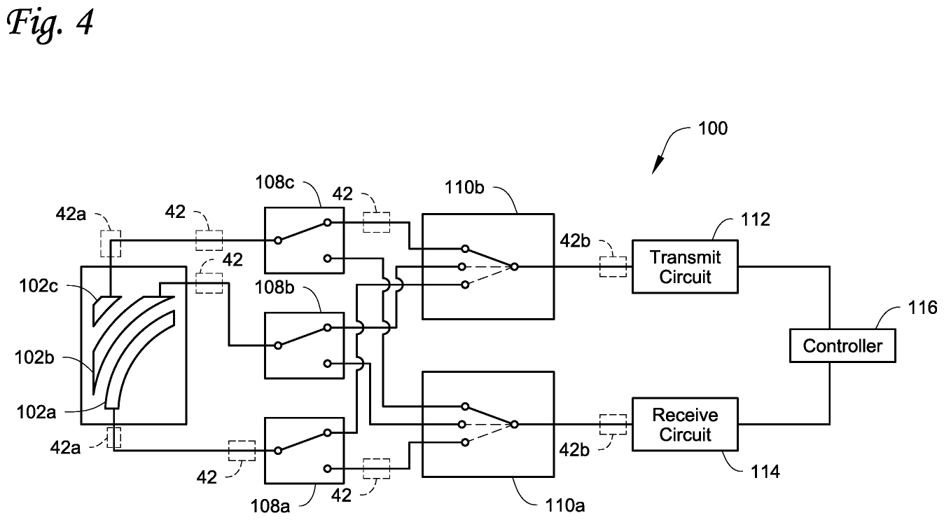

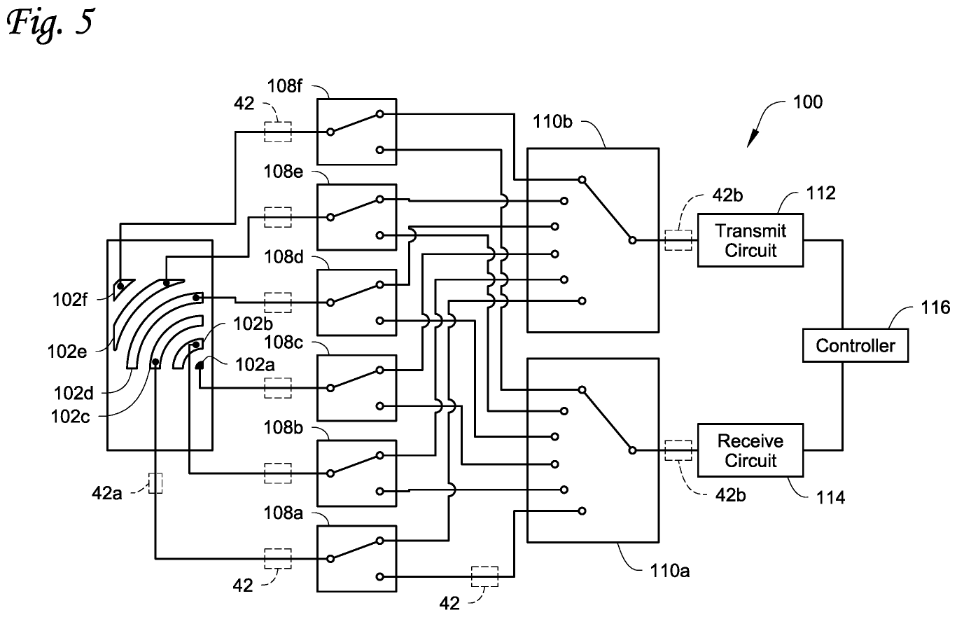

FIGS. 4-5 are schematic depictions of additional embodiments of a non-invasive analyte sensor system 100. The systems 100 depicted in FIGS. 4-5 includes at least three or more antennas (FIG. 4) or at least six or more antennas (FIG. 5). However, a different number of antennas can be used. In each of the embodiments, the system 100 is configured so that one or more of the antennas of the antenna array can be used as either a transmit antenna or as a receive antenna. In FIGS. 4-5, like elements are referenced using the same reference numerals. As with the previously described embodiment in FIG. 1, the antenna arrays in FIGS. 4-5 can be a decoupled antenna array and the antennas of the antenna array can be decoupled from one another. However, in some embodiments, the antennas of the system 100 may not be decoupled from one another. In one embodiment, the antennas used in the arrays in FIGS. 4-5 can have different geometries from each other.

In the embodiment in FIG. 4, the antenna array of the system 100 has three antennas 102a, 102b, 102c each of which is disposed on a substrate 106. The system further includes three switches 108a, 108b, 108c, a receive switch controller 110a, a transmit switch controller 110b separate from the receive switch controller 110a, a transmit circuit 112, a receive circuit 114, and a controller 116. In the embodiment in FIG. 5, the antenna array of the system 100 has six antennas 102a-f each of which is disposed on the substrate 106, and six of the switches 108a-f. Further information on the system 100 in FIGS. 4 and 5 can be found in U.S. Pat. No. 11,058,321, the entire contents of which are incorporated herein by reference.

In the systems in FIGS. 4 and 5, the noise reduction components 42, 42a, 42b (shown in dashed lines) can be provided at one or more locations on the electrical conductors that form the signal paths between the antennas 102a-c and the receive circuit 114 and/or the transmit circuit 112. The noise reduction components 42 can be located around the electrical conductors similar to the embodiments in FIGS. 2-3, and the components 42a, 42b can be disposed around RF adapters (not shown for convenience) that function similarly to the RF adapters 44a, 44b in FIGS. 2-3.

The terminology used in this specification is intended to describe particular embodiments and is not intended to be limiting. The terms “a,” “an,” and “the” include the plural forms as well, unless clearly indicated otherwise. The terms “comprises” and/or “comprising,” when used in this specification, specify the presence of the stated features, integers, steps, operations, elements, and/or components, but do not preclude the presence or addition of one or more other features, integers, steps, operations, elements, and/or components.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A non-invasive analyte sensor system, comprising:
a first antenna that is configured to emit radio frequency electromagnetic waves, the first antenna is positioned and arranged to transmit a radio frequency transmit signal into a target containing at least one analyte;
a second antenna that is configured to detect radio frequency electromagnetic waves, the second antenna is positioned and arranged to detect a radio frequency response resulting from transmission of the radio frequency transmit signal by the first antenna into the target containing the at least one analyte;
a transmit circuit that is electrically connectable to the first antenna, the transmit circuit is configured to generate the radio frequency transmit signal to be transmitted by the first antenna;
a transmit electrical conductor electrically connecting the transmit circuit with the first antenna;
a receive circuit that is electrically connectable to the second antenna, the receive circuit is configured to receive the radio frequency response detected by the second antenna;
a receive electrical conductor electrically connecting the receive circuit with the second antenna; and
at least one noise reducer on the receive electrical conductor between the receive circuit and the second antenna and/or on the transmit electrical conductor between the transmit circuit and the first antenna.

2. The non-invasive analyte sensor system of claim 1, wherein the at least one noise reducer is on the receive electrical conductor between the receive circuit and the second antenna.

3. The non-invasive analyte sensor system of claim 2, comprising a first radio frequency adapter connecting the receive electrical conductor to the second antenna and a second radio frequency adapter connecting the receive electrical conductor to the receive circuit, and the at least one noise reducer surrounds the first radio frequency adapter or the second radio frequency adapter.

4. The non-invasive analyte sensor system of claim 1, wherein the at least one noise reducer comprises a choke.

5. The non-invasive analyte sensor system of claim 1, wherein the at least one analyte comprises glucose, alcohol, white blood cells, or luteinizing hormone.

6. A non-invasive analyte sensor system, comprising:
an antenna array having at least three antennas each of which is configured to emit and receive radio frequency electromagnetic waves;
a transmit circuit that is selectively electrically connectable to any one or more of the at least three antennas, the transmit circuit is configured to generate at least one transmit signal in a radio frequency range of the electromagnetic spectrum to be transmitted into a target by the one or more of the at least three antennas the transmit circuit is electrically connected to;
a receive circuit that is selectively electrically connectable to any one or more of the at least three antennas, the receive circuit is configured to receive a response detected by the one or more of the at least three antennas the receive circuit is electrically connected to resulting from transmission of the at least one transmit signal into the target containing the at least one analyte of interest;
electrical conductors electrically connecting the receive circuit with the at least three antennas;
noise reducers on the electrical conductors between the receive circuit and the at least three antennas.

7. The non-invasive analyte sensor system of claim 6, wherein the noise reducers comprise chokes.

8. The non-invasive analyte sensor system of claim 6, comprising first radio frequency adapters connecting the electrical conductors to the antennas and second radio frequency adapters connecting the electrical conductors to the receive circuit, and the noise reducers surround the first radio frequency adapters or the second radio frequency adapters.

9. The non-invasive analyte sensor system of claim 6, wherein the at least one analyte comprises glucose, alcohol, white blood cells, or luteinizing hormone.
